(19) United States
(12) Patent Application Publication
(10) Pub. No.: US 2009/0182251 A1
(43) Pub. Date: Jul. 16, 2009

(54) COVERS FOR ORTHOPEDIC CASTS

(76) Inventor: Mark Alan Bennett, Broadlands, VA (US)

Correspondence Address: STEPHEN CHRISTOPHER SWIFT;SWIFT LAW OFFICE, SUITE 600, 1800 DIAGONAL ROAD, ALEXANDRIA, VA, 22314-2840, US

(21) Appl. No.: 12/014,748

(22) Filed: Jan. 15, 2008

(51) Int. Cl. A61F 5/00

(52) U.S. Cl. 602/3; 602/36

(57) ABSTRACT

Cast covers, that allow someone who has a casted arm or leg, and has been authorized by a physician to resume playing a particular sport (e.g., football), to protect both the casted limb and other players from collision with the hard material in the cast, by covering the cast with a cushioning material. The cast covers are precast and premolded in different sizes and colors to meet individual needs and tastes. The cast cover has a generally tubular shape. Preferably, a thick inner layer of polyurethane foam is covered by a thinner outer layer of polyethylene foam. In a first embodiment, the tube is closed, except for openings at its ends. In a second embodiment, there is a longitudinal opening running between the open ends of the tube, and the tube is secured on a casted limb by strips of hook and loop fasteners that traverse the longitudinal opening.

### BACKGROUND OF THE INVENTION

1. Field of the Invention

The present invention relates to covers for orthopedic casts.

2. Description of the Prior Art

There are prior inventions of covers for orthopedic casts, but none that are equivalent to the present invention.

U.S. Pat. No. 3,416,518, issued on Dec. 17, 1968, to Bessie M. Samuels and Edith M. Long, discloses a cast cover for an arm or a leg, but does not disclose the two layers of foam of the instant invention.

U.S. Pat. No. 4,646,727, issued on Mar. 3, 1987, to David H. Chambers, discloses a leg cast cover, but does not disclose the two layers of foam of the instant invention.

U.S. Pat. No. 4,986,265, issued on Jan. 22, 1991, to Ronald E. Caponi, discloses a protective cover for a cast, with hook and loop fasteners, but without the two layers of foam of the instant invention.

U.S. Pat. No. 5,070,630, issued on Dec. 10, 1991, to Ross Edmundson, discloses a decorative cast cover, with only a single layer of polystyrene foam.

U.S. Pat. No. 5,643,183, issued on Jul. 1, 1997, to Joseph C. Hill, discloses waterproof cover for casts and bandages, without the two foam layers of the instant invention.

U.S. Pat. No. 5,720,712, issued on Feb. 24, 1998, to Dave A. Joy and Robert B. Gordon, discloses a reusable limb protector to protect a limb with a cast or bandage, without the two foam layers of the instant invention.

U.S. Pat. No. 6,047,403, issued on Apr. 11, 2000, to Penny Juozaitis, discloses a decorative cast covering, without any foam layer.

U.S. Pat. No. 6,126,621, issued on Oct. 3, 2000, to Santiago Aceves, discloses a cast covering apparatus, without the two foam layers of the instant invention.

U.S. Pat. No. 6,932,784, issued on Aug. 23, 2005, to Debra J. Reading, discloses a protective and insulating cover for an injured limb, having a double layer of material only at the toes.

U.S. Pat. No. 7,020,899, issued on Apr. 4, 2006, to Frank Vincent Carlopio, discloses fluid impervious cast protector, without the two foam layers of the instant invention.

U.S. Pat. No. 7,066,899, issued on Jun. 27, 2006, to Sally J. Baron, discloses a cast cover with spaces for fingers or toes, without the two foam layers of the instant invention.

U.S. Pat. No. Des. 323,217, issued on Jan. 14, 1992, to Katherine A. Holden, discloses a design for an arm cast cover. Neither this design patent, nor any of the following design patents, disclose the two foam layers of the instant invention.

U.S. Pat. No. Des. 406,897, issued on Mar. 16, 1999, to Elizabeth Agati and David Agati, discloses a design for a waterproof cast protector.

U.S. Pat. No. Des. 410,088, issued on May 18, 1999, to Paula J. Rutledge and Larry R. Rutledge, discloses a design for a cast cover for a leg, having two pockets.

U.S. Pat. No. Des. 415,282, issued on Oct. 12, 1999, to Paula J. Rutledge and Larry R. Rutledge, discloses a design for a cast cover for an arm, having two pockets.

U.S. Pat. No. D483,492, issued on Dec. 9, 2003, to Sunny Dumani, discloses a design for a cast cover with wavy diagonal stipes.

U.S. Pat. No. D484,604, issued on Dec. 30, 2003, to Debbie K. Tramel et al., discloses a design for a decorative cast cover, with smiley faces.

U.S. Pat. No. D545,442, issued on Jun. 26, 2007, to Michael R. Weeks, discloses a design for a waterproof cast protector.

U.S. Pat. No. D549,348, issued on Aug. 21, 2007, to Scott M. Przybycien, discloses a design for a cast cover, with what appear to be strips of hook and loop fasteners.

U.S. Patent Application Publication No. 2001/0041853, published on Nov. 15, 2001, to Robert W. South and Olivia South, discloses a protective cast cover, without the two foam layers of the instant invention.

U.S. Patent Application Publication No. 2003/0191419, published on Oct. 9, 2003, to Mark Melin and Cindy Melin, discloses a protective cover for a cast, without the two foam layers of the instant invention.

U.S. Patent Application Publication No. 2006/0287623, published on Dec. 21, 2006, to Kent F. Beck and Timothy P. Walker, discloses a protective cast cover. FIG. 7 and paragraph 0040 on page 3 disclose that the cast cover may have two layers. However, it is not disclosed that the two layers are made of foam, as in the instant invention.

Japanese Patent No. 2004-160127, published on Jun. 10, 2004, to Hideki Tsunoda, discloses a plaster cast cover, that is easy to attach or remove.

None of the above inventions and patents, taken either singly or in combination, is seen to describe the instant invention as claimed.

### SUMMARY OF THE INVENTION

The present invention is cast covers, that allow someone who has a casted arm or leg, and has been authorized by a physician to resume playing a particular sport (e.g., football), to protect both the casted limb and other players from collision with the hard material in the cast, by covering the cast with a cushioning material. The cast covers are precast and premolded in different sizes and colors to meet individual needs and tastes. The cast cover has a generally tubular shape. Preferably, a thick inner layer of polyurethane foam (such as PPT foam) is covered by a thinner outer layer of polyethylene foam (such as PLASTAZOTE Polyethylene Foam). In a first embodiment, the tube is closed, except for openings at its ends. In a second embodiment, there is a longitudinal opening running between the open ends of the tube, and the tube is secured on a casted limb by strips of hook and loop fasteners that traverse the longitudinal opening. In a third embodiment, one end partially covers a hand, with a small opening for the thumb. In a fourth embodiment, there is a pair of T-shaped strips with hook and loop fasteners, where the tops of the T's can be joined to form a loop around the limb adjacent to one opening in the end of the cast cover, while the bottoms of the T's are fastened to sides of the tube.

Accordingly, it is a principal object of the invention to reduce the risk of injuries to other participants when someone with a cast participates in athletic activities.

It is another object of the invention to reduce the risk of further injury to a person wearing a cast when they participate in athletic activities.

It is a further object of the invention to reduce the risk of damage to a cast when the person wearing the cast participates in athletic activities.

Still another object of the invention is to enable a person wearing a cast to participate in a variety of activities.

It is an object of the invention to provide improved elements and arrangements thereof in an apparatus for the purposes described which is inexpensive, dependable and fully effective in accomplishing its intended purposes.

These and other objects of the present invention will become readily apparent upon further review of the following specification and drawings.

### BRIEF DESCRIPTION OF THE DRAWINGS

FIG. 1 is a perspective view of the first preferred embodiment of the invention.

FIG. 2 is a sectional view drawn along lines 2-2 of FIG. 1.

FIG. 3 is a perspective view of the second preferred embodiment of the invention.

FIG. 4 is a perspective view of the third preferred embodiment of the invention.

FIG. 5 is a perspective view of the fourth preferred embodiment of the invention, with the T-shaped straps in an open position.

FIG. 6 is a perspective view of the fourth preferred embodiment of the invention, with the T-shaped straps in a closed position.

Similar reference characters denote corresponding features consistently throughout the attached drawings.

### DETAILED DESCRIPTION OF THE PREFERRED EMBODIMENTS

The present invention is protective covers for orthopedic casts. Preferably, they are precast and/or pre-molded in different sizes and colors to meet individual needs. Alternatively, they may be custom made to fit a particular cast.

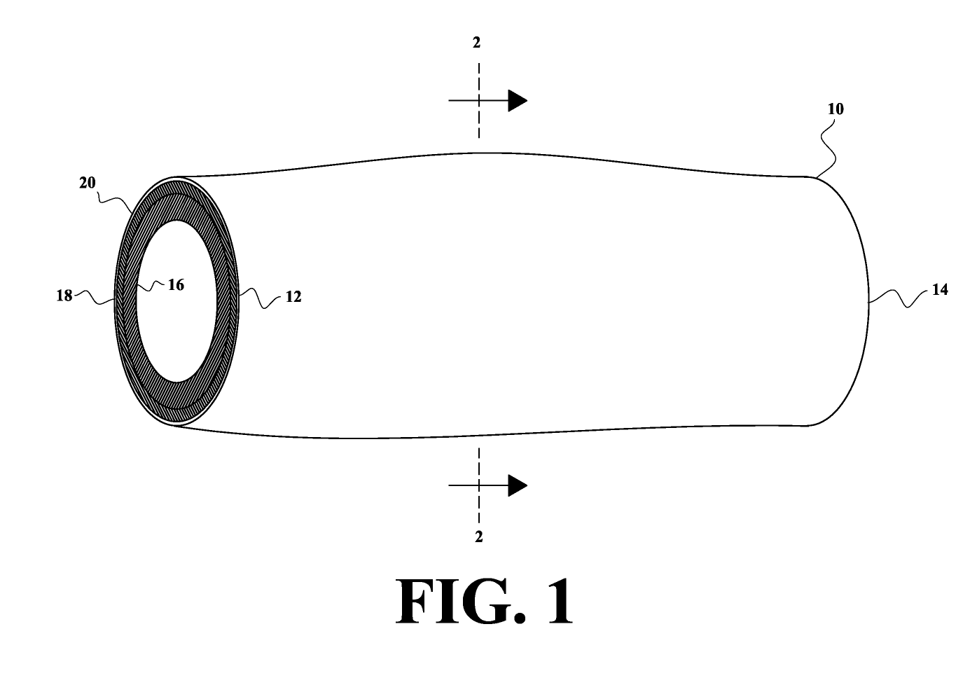

FIG. 1 is a perspective view of the first preferred embodiment of the invention, showing the cast cover 10, having openings at each of two opposite ends 12 and 14, suitably dimensioned and configured to enable it to be inserted over a limb covered by an orthopedic cast. The cast cover has an inner layer 16 of foam material that is solid but elastic, with the inner layer having an inner surface that is suitably dimensioned and configured to entirely cover the outer surface of an orthopedic cast, and an outer layer 18 of foam material that is solid but elastic, with the outer layer being adjacent to and covering the inner layer. The outer layer may be covered by a protective layer 20 of flexible non-foam material, such as natural or synthetic cloth or leather. Preferably, the inner layer is thicker than the outer layer. The foam material should be a high energy absorbing material that will not “bottom out”. Preferably, the inner layer is made of polyurethane foam (such as PPT foam) and the outer layer is made of polyethylene foam (such as PLASTAZOTE Polyethylene Foam) Alternatively, both the inner and outer layer may be made of polyurethane foam. Spray-on adhesive may be used to join the inner and outer layers. Baby powder may make it easier to insert the cover over the cast.

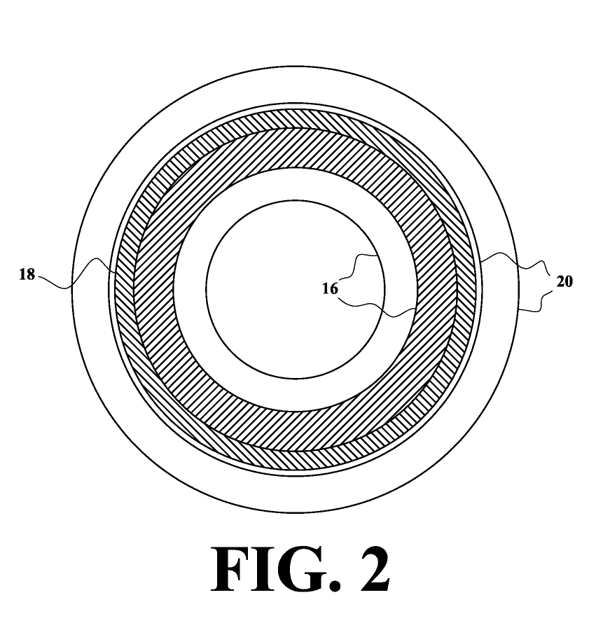

FIG. 2 is a sectional view drawn along lines 2-2 of FIG. 1, showing the inner layer 16, the outer layer 18, and the protective layer 20.

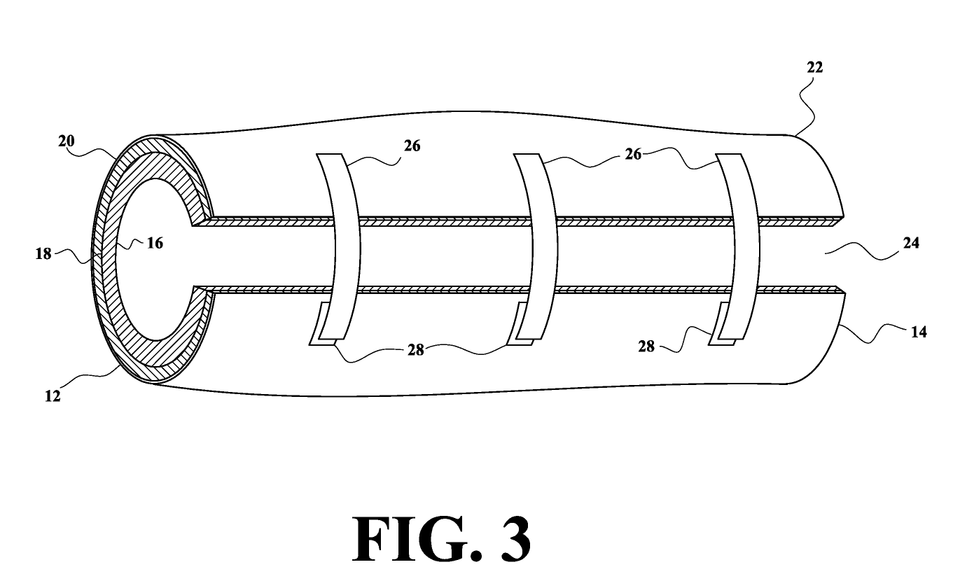

FIG. 3 is a perspective view of the second preferred embodiment of the invention, which is the same as the first preferred embodiment, except that the cast cover 22 has a longitudinal opening 24, with strips 26 having hook and loop fasteners that can traverse the longitudinal opening and be joined to pads 28 having hook and loop fasteners, to secure the cover on an orthopedic cast.

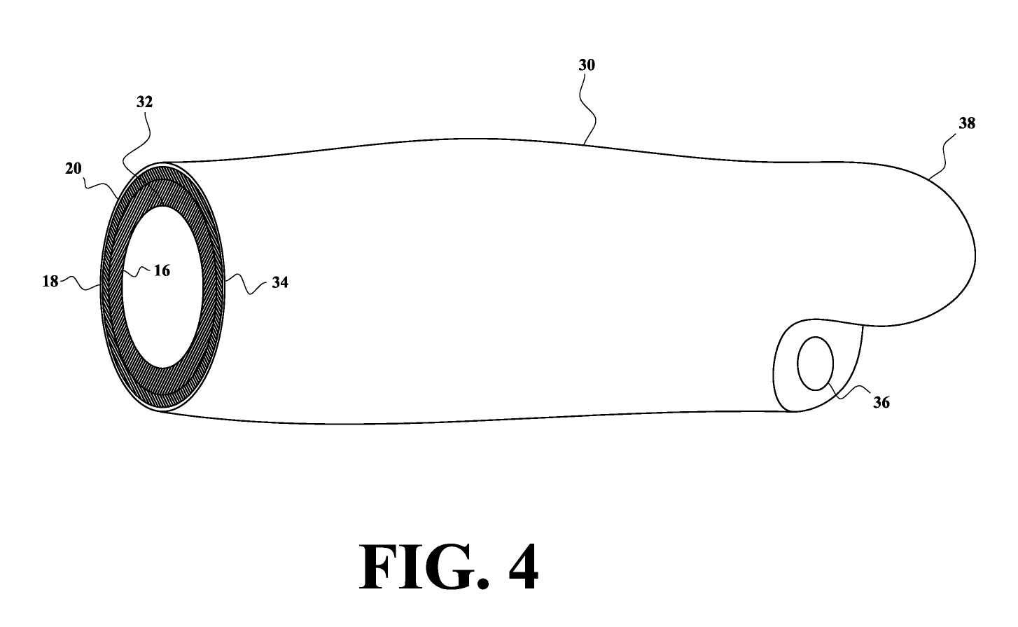

FIG. 4 is a perspective view of the third preferred embodiment of the invention, which is the same as the first preferred embodiment, except that the cast cover 30 has one opening 32 at a first end 34, suitably dimensioned and configured to enable it to be inserted over a hand, and a smaller opening 36 for a thumb at a second end 38. The second end is mainly closed to cover the rest of the hand.

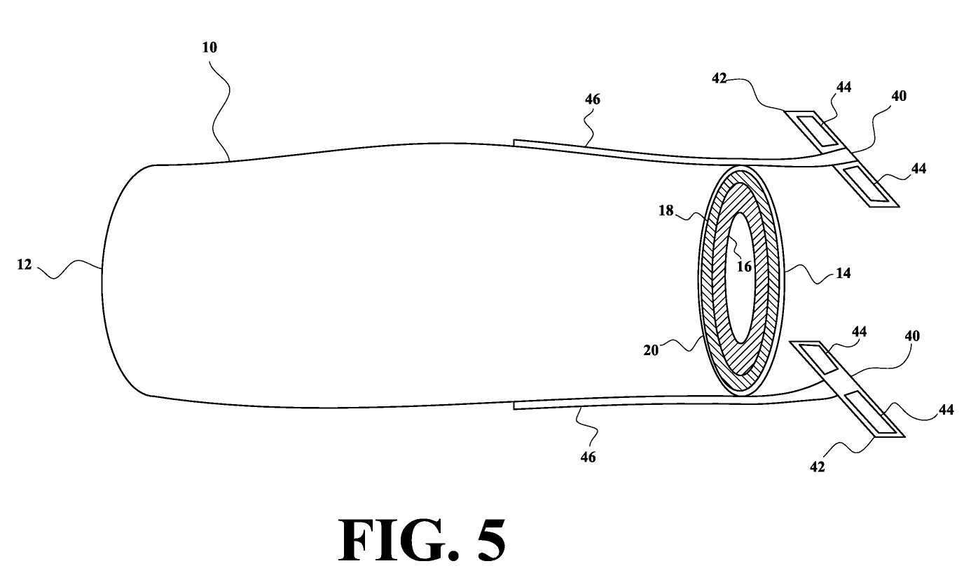

The fourth preferred embodiment is the same as the first preferred embodiment, except that it has T-shaped straps extending from one end of the cast cover. FIG. 5 is a perspective view of the fourth preferred embodiment of the invention, with the pair of T-shaped straps 40 in an open position. The T-shaped strips have top members 42 with hook and loop fasteners 44, and bottom members 46 that are fastened to sides 48 of the cast cover.

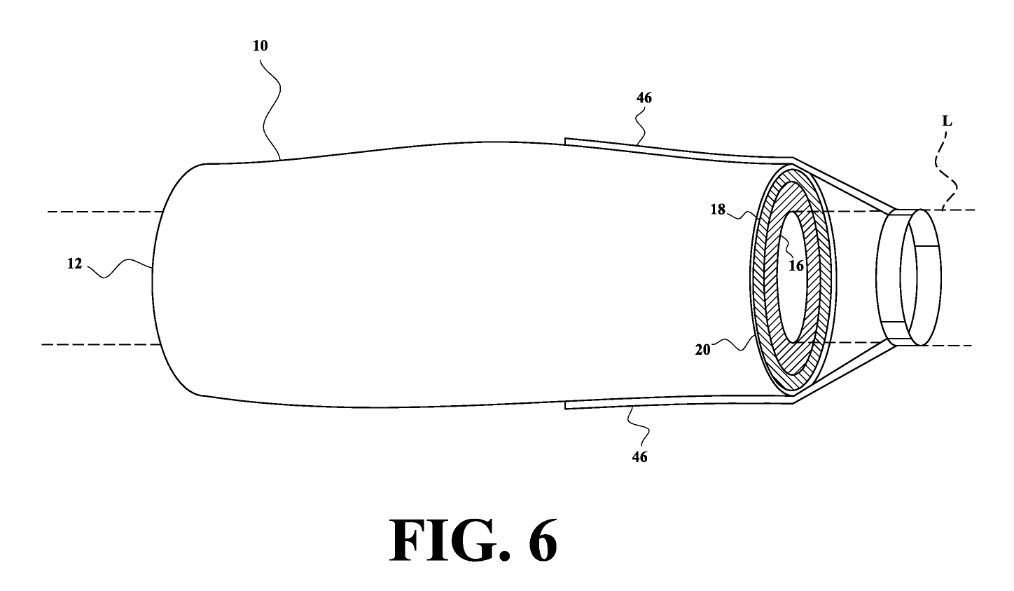

FIG. 6 is a perspective view of the fourth preferred embodiment of the invention, with the T-shaped straps in a closed position, in which they are joined to form a loop 50 around a limb L adjacent to the opening in the end 14 of the cast cover.

It is to be understood that the present invention is not limited to the embodiments described above, but encompasses any and all embodiments within the scope of the following claims.

I claim:

1. A cover for an orthopedic cast, comprising:
an inner layer of foam material that is solid but elastic, with the inner layer having an inner surface that is suitably dimensioned and configured to entirely cover the outer surface of an orthopedic cast; and
an outer layer of foam material that is solid but elastic, with the outer layer being adjacent to and covering the inner layer.

2. The cover for an orthopedic cast according to claim 1, wherein:
the inner layer is made of polyurethane foam; and
the outer layer is made of polyethylene foam.

3. The cover for an orthopedic cast according to claim 1, wherein:
the inner layer and the outer layer are both made of polyurethane foam.

4. The cover for an orthopedic cast according to claim 1, wherein:
the outer layer is covered by a protective layer of flexible non-foam material.

5. The cover for an orthopedic cast according to claim 1, wherein:
the cover has an opening at each of two opposite ends, suitably dimensioned and configured to enable it to be inserted over a limb covered by an orthopedic cast.

6. The cover for an orthopedic cast according to claim 1, wherein:
the cover has one opening at a first end, suitably dimensioned and configured to enable it to be inserted over a hand, and a smaller opening for a thumb at a second end.

7. The cover for an orthopedic cast according to claim 1, further comprising:
a pair of T-shaped strips with hook and loop fasteners, said strips having top members that can be joined to form a loop around a limb adjacent to an opening in an end of the cover, and bottom members that are fastened to sides of the cover.

8. A cover for an orthopedic cast, comprising:
an inner layer of foam material that is solid but elastic, with the inner layer having an inner surface that is suitably dimensioned and configured to cover a portion of the outer surface of an orthopedic cast; and
an outer layer of foam material that is solid but elastic, with the outer layer being adjacent to and covering the inner layer.

9. The cover for an orthopedic cast according to claim 8, wherein:
the inner layer is made of polyurethane foam; and
the outer layer is made of polyethylene foam.

10. The cover for an orthopedic cast according to claim 8, wherein:
the inner layer and the outer layer are both made of polyurethane foam.

11. The cover for an orthopedic cast according to claim 8, wherein:
the outer layer is covered by a protective layer of flexible non-foam material.

12. The cover for an orthopedic cast according to claim 8, wherein:
the cover has an opening at each of two opposite ends, suitably dimensioned and configured to enable it to be inserted over a limb covered by an orthopedic cast.

13. The cover for an orthopedic cast according to claim 8, wherein:
the cover has one opening at a first end, suitably dimensioned and configured to enable it to be inserted over a hand, and a smaller opening for a thumb at a second end.

14. The cover for an orthopedic cast according to claim 8, wherein:
there is a longitudinal opening running between open ends of the cover, with strips having hook and loop fasteners that can traverse the longitudinal opening and be joined pads having hook and loop fasteners, to secure the cover on an orthopedic cast.

15. A method of making a cover for an orthopedic cast, comprising the steps of:
forming an inner layer of foam material that is solid but elastic, with the inner layer having an inner surface that is suitably dimensioned and configured to entirely cover the outer surface of an orthopedic cast; and
covering the inner layer with an outer layer of foam material that is solid but elastic.

16. The method of making a cover for an orthopedic cast according to claim 15, wherein:
the inner layer is made of polyurethane foam; and
the outer layer is made of polyethylene foam.

17. The method of making a cover for an orthopedic cast according to claim 15, wherein:
the inner layer and the outer layer are both made of polyurethane foam.

18. The method of making a cover for an orthopedic cast according to claim 15, further comprising the step of:
covering the outer layer by a layer of flexible non-foam material.

19. The method of making a cover for an orthopedic cast according to claim 15, wherein:
the cover has an opening at each of two opposite ends, suitably dimensioned and configured to enable it to be inserted over a limb covered by an orthopedic cast.

20. The method of making a cover for an orthopedic cast according to claim 15, wherein:
the cover has one opening at a first end, suitably dimensioned and configured to enable it to be inserted over a hand, and a smaller opening for a thumb at a second end.
